(19) United States
(12) Patent Application Publication
(10) Pub. No.: US 2006/0156953 A1
(43) Pub. Date: Jul. 20, 2006

(54) Mixture for preparing water-based hardenable mixings intended to realise mainly soundroofing agglomerates and method for preparing said mixings

(76) Inventor: Gianfranco Toscano, Venaria Reale (Torino) (IT)

Correspondence Address: BACON & THOMAS, PLLC, 625 SLATERS LANE, FOURTH FLOOR, ALEXANDRIA, VA, 22314, US

(21) Appl. No.: 10/563,769

(22) PCT Filed: Jul. 2, 2004
(86) PCT No.: PCT/IB04/02213

§ 371 Date: Jan. 9, 2006

(30) Foreign Application Priority Data
Jul. 7, 2003 (EP) 03425446.6

(51) Int. Cl. A61L 15/32; C08L 97/02; C08L 1/00; C04B 20/00; C04B 16/00; C04B 24/00; C04B 14/00; C04B 2/00; C08L 5/00

(52) U.S. Cl. 106/122; 106/163.01; 106/162.5; 106/712; 106/674; 106/730; 106/731; 106/795; 366/348; 366/1

(57) ABSTRACT

The invention relates to a mixture containing a building binder (preferably cement), comprising cellulose, ready for preparing water-based hardenable mixings intended to realise mainly soundproofing agglomerates suitable for laying or for producing manufactured articles or for fabricating structural building elements or for other engineering works. The invention further relates to a method for preparing said hardenable mixings. The invention further relates to a mainly soundproofing non structural agglomerate obtained from said mixture when a “light inert” is added and to a structural mainly soundproofing agglomerate obtained from said mixture when an “heavy inert” is added.

### DISCLOSURE OF THE INVENTION

The present invention refers to mixtures containing a building binder (cement or the like), inerts and cellulose.

More particularly, the present invention refers to mixtures for preparing water-based hardenable mixings intended to produce mainly soundproofing agglomerates suitable for laying or for producing manufactured articles in the building field or even for fabricating structural building elements such as columns, beams and floor slabs, or for other engineering works, said agglomerates having reduced expansion resulting from their soundproofing and heat insulating properties.

The invention further concerns a method for preparing said hardenable mixings, said method being also applicable in fields different from the building field.

The invention further regards a cement-based mainly soundproofing non structural agglomerate obtained from said mixture.

The invention further regards a cement-based structural mainly soundproofing agglomerate obtained from said mixture.

Cement-based mixtures are known, containing additional materials of different kinds contributing to the structure thermal and acoustic insulating properties, that are employed in the building field mainly in the granular form such as filling for floor foundations or, in the form of panels, bricks or tiles, such as wall covers.

Said additional materials, besides having thermal and acoustic insulating properties, must also combine lightness and compression resistance features; furthermore, a sufficient hardness has to be maintained, depending on the manufacturing type and therefore being a non structural hardness.

Needless to say, then, that it is a basic practice in the building field that cement, or another building binder such as lime or any natural or synthetic binder acting as a cement-like binder, is mixed with a fine inert aggregate (for instance, sand) and water to make mortar, said mortar being mixed, in its turn, with a coarse inert aggregate (for instance, broken stones) to make concrete typically used for fabricating structural building elements such as columns, beams and floor slabs; it is well known that the concrete has a high thermal and acoustic conductivity.

According to the common knowledge in the building field, the additional materials referred to as hereinabove can be roughly divided into to main categories: the “light inerts” having a specific weight in the range from 0 kg/m³ to about 500 kg/m³ and the “heavy inerts” having a specific weight in the range from about 500 kg/m³ to about 2,000 kg/m³. This classification is useful for many purposes in the building field; its use for the purposes of the present invention will become more clear hereinafter.

It is well known by all the persons operating in the field that all the thermal insulating materials are also partially acoustic insulating materials; it therefore results that all the acoustic insulating materials are also thermal insulating materials. This clarification is useful to highlight that the present invention improves the thermal insulating values and produces definitely high noise reduction values in comparison with the values of the current products: for this reason, the agglomerates according to the present invention are referred to as mainly soundproofing agglomerates.

Mixtures of the above-mentioned type, i.e. giving the final product soundproofing properties, are broadly widespread and employed in the building field as well as widely known from the prior art in said reference technical field.

For instance, the European patent application no. 0 041 053 published on Dec. 2, 1981 discloses a light soundproofing material, in the form of pebbles or balls, obtained by thoroughly mixing powdery or granular soundproofing ingredients among them and, preferably but not compulsorily, with inert powders, bonding the resulting mixture together by cement substantially in the dry state, and then enclosing the final cemented mixture in a hard cement crust; the method for manufacturing said light soundproofing material, in the form of pebbles or balls, is also therein disclosed.

As regards the aforementioned U.S. patent, it is worthy to note that it is specifically directed to provide soundproofing materials in the forms of pebbles or balls, i.e. materials either to be employed as such in their dry state or to be agglomerated with binding materials such as cement, and presumably water, to form light castings.

In accordance with the foregoing, said pebbles or balls have to be considered as a raw material, rather different from an intermediate mixture ready to be used, with the following drawback that inhomogeneous mixings are produced most likely having inhomogeneous soundproofing properties.

Another example can be found in the U.K. patent no. 297,750 granted on May 30, 1929, which discloses soundproofing floor covering compositions as such, i.e. already comprising water.

In accordance with the foregoing, said floor covering compositions have to be considered as a final product, rather different from an intermediate mixture ready to be used, with the following drawback that they can be applied only in the application for they are provided and they can not be applied for structural purposes; furthermore, said floor covering compositions provide lightness and thermal insulating properties but they are most likely not able to give a high noise reduction.

A further example can be found in the U.S. Pat. No. 6,475,275 granted on Nov. 5, 2002, which discloses compositions comprising cement, clay, an aggregate and/or a fibrous material, and a retarder, said compositions being provided with the main purpose to slow setting process in slurries of the cement composition thanks to the retarder presence; furthermore other components are provided such as, for instance, an accelerating agent.

In accordance with the foregoing, said compositions can be considered as intermediate mixtures ready to be used, but the need to contemporaneously provide so many ingredients results in very costly as well as very laborious (as far as the manufacturing process is concerned) compositions, while achieving a low noise reduction.

The main purpose of the present invention is to provide a mixture containing a building binder, preferably a cement-based mixture, that is ready to be used and that, suitably water-supplemented as well as added up with “light inerts”, allows to obtain an agglomerate having improved soundproofing properties and similar thermal, lightness and step-steadiness properties in comparison with the ones reachable by mixtures according to the known prior art.

A further purpose of the present invention is to provide a mixture containing a building binder, preferably a cement-based mixture, that is ready to be used and that, suitably water-supplemented as well as added up with “heavy inerts”, allows to obtain a structural agglomerate having high compression resistance properties in addition to the soundproofing and heat insulating properties, such agglomerates being usable for fabricating structural building elements that contribute too to the overall mainly soundproofing properties.

A further purpose of the present invention is to provide a mixture containing a building binder, preferably a cement-based mixture, that employs not many, easily available and not particularly valuable materials, the mixture thus resulting to be rather practical and economical.

The aforementioned and further purposes of the invention are achieved by the cement mixture as well as by the method as defined in the attached claims.

Thanks to the cement mixture according to the invention, which provides for the addition of cellulose, the advantage of obtaining an improvement of the agglomerate soundproofing properties is achieved, said agglomerate being either laid at the liquid state onto floor foundations or employed both for manufacturing products of any shape and size, such as panels, bricks and tiles, and for fabricating structural building elements such as columns, beams and floor slabs.

More particularly, thanks to the cement mixture according to the invention, which provides for the addition of cellulose besides the additional material ranging from “light inerts” to “heavy inerts”, the advantage of obtaining a wide range of soundproofing agglomerates, from agglomerates having soundproofing properties only up to agglomerates combining soundproofing and structural properties, is achieved.

Furthermore, advantageously, a light and nevertheless mechanically resistant agglomerate is produced.

Furthermore, advantageously, by employing easily available and not particularly valuable materials, the agglomerate proves to be rather practical and economical.

As far as the manufacturing method is concerned, it also allows to optimise operational times and costs by using a conventional building concrete-mixer fit for continuous operation.

Further features and advantages will become evident from the description of a preferred embodiment of the present invention, which will be better disclosed with reference to a sample case.

The cement mixture according to the invention, suitable for preparing water-based hardenable mixings intended to produce mainly soundproofing agglomerates, is obtained by adding some cellulose and inert to the cement and water.

The mixture comprises a cellulose amount that varies on the basis of both the cement amount and the properties of the inert employed, said cellulose amount preferably ranging from 5 kg for each cubic meter of finished material up to a maximum of 100 kg for each cubic meter of finished material.

The cellulose employed can be in the form of both fibres and paper or cardboard or the like, even recycled.

In case cellulose is in the form of fibres, said fibres will preferably have a length in the range of 0.05÷10 mm and a diameter in the range of 0.05÷10 mm.

In case cellulose is in the form of paper products, even recycled, the pieces of said paper and/or cardboard products will preferably have size less than 100 mm².

Preferably, cellulose is added only after that a portion of cement has been homogeneously mixed with water, before inerts addition.

Besides cellulose, in the mixture can be optionally added some cork, which helps to mix the mixture components and which can be in the form of both granules and powder; in case cork granules are used, they will preferably have size less than 100 mm².

Cork, even not being an essential component, allows to increase the value of noise reduction; on the basis of the experimental tests carried out, it has been noticed that, thanks to the cork presence, the noise reduction value has increased by about 3 dB.

In the mixture can be used some polystyrene or similar materials, either virgin or recycled, preferably in spherical form or ground, as inert in order to obtain the wanted volume.

Polystyrene is added in order to introduce air or as a “light inert”.

Alternatively, besides cellulose, and in case also additionally to cork, air can be fed in the form of binder-compatible foam (like soap or another commercial foam) or any other air-carrier “light inert” having a specific weight in the range from 0 kg/m³ to about 500 kg/m³ can be used in case a mixture suitable for preparing water-based hardenable mixings intended to produce agglomerates having mainly soundproofing properties only (i.e. not structural) is to be obtained.

In case a mixture suitable for preparing water-based hardenable mixings intended to produce structural mainly soundproofing agglomerates is to be obtained, “heavy inerts” having a specific weight in the range from about 500 kg/m³ to about 2,000 kg/m³, such as sand, gravel, pebbles, river shingle, broken stones or bricks and the like, are added.

The ratio of cement to “light inert” or “heavy inert”, can vary so as to obtain an agglomerate density preferably varying between 100 kg/m³ (value below which the obtained covering has an insufficient compression resistance and it is no more step-steady) and 600 kg/m³ (value above which there is no more the beneficial economical result).

Besides cellulose, in the mixture can be optionally added a coloured pigment, preferably iron oxide, that will give the mixture a typical colour.

The coloured pigment is added in order to give the agglomerate a colour such as to differentiate the new agglomerate with respect to the conventional cement materials; in case iron oxide is added, the colour will be reddish.

Upon the cement-based mainly soundproofing agglomerate obtained starting from the mixture according to the invention, laid onto a floor foundation, can be further placed a layer of tarred paper, acting as a separation intermediate layer and facilitating the noise reduction thanks to the direct conductivity; said layer of tarred paper has preferably a thickness of 0.5 mm.

Alternatively, upon the agglomerate can be placed a nylon layer acting as a separation intermediate layer.

Upon the arrangement so realised and, particularly, upon the intermediate layer, of tarred paper or nylon or the like, a floor foundation can be placed.

The method according to the invention for preparing water-based hardenable mixings intended to produce mainly soundproofing agglomerates starting from a cement mixture, comprises the steps of:

preparing proportionate amounts of the different components (cement, cellulose, inerts and water) depending on the final structural or non structural application;

mixing said components till obtaining a hardenable mixing suitable for laying or for producing manufactured articles or for fabricating structural building elements such as columns, beams and floor slabs.

Since the method according to the invention employs a technology known to a person skilled in the field, said technology will not be disclosed in detail.

Besides the components mentioned above, a certain quantity of air is introduced by force inside said mixing; said air quantity can be conveyed by a foam or, alternatively, by a spongy material such as, for instance, ground polystyrene.

In case air is conveyed by means of foam, said foam is preferably a commercial foam or a foam obtainable either by transforming a foaming liquid product or by mixing soap with water.

Alternatively, any other air-carrier “light inert” having a specific weight in the range from 0 kg/m³ to about 500 kg/m³ can be used in case a mixture suitable for preparing water-based hardenable mixings intended to produce agglomerates having mainly soundproofing properties only is to be obtained.

In case a mixture suitable for preparing water-based hardenable mixings intended to produce structural mainly soundproofing agglomerates is to be obtained, “heavy inerts” having a specific weight in the range from about 500 kg/m³ to about 2,000 kg/m³, such as sand, gravel, pebbles, river shingle, broken stones or bricks and the like, are added.

Optionally, a certain quantity of cork pieces or powder can be inserted.

Optionally, a certain quantity of a coloured pigment can be further inserted.

The method according to the invention is carried out by using a building concrete-mixer or any other mixing apparatus; preferably, said concrete-mixer is started-up before introducing the raw materials and it is continuously operated (i.e. the concrete-mixer starts working from the introduction of water), this helping to achieve a better agglomerate mixing, which is liquid when laid onto floor foundations or during the manufactured articles production.

The components constituting the agglomerate are inserted in accordance with the following chronological sequence: water; cement; cellulose and inerts.

Moreover, experimental tests have been carried out at a certified Institute having shown that a significant noise reduction is attained by a soundproofing agglomerate according to the invention with density in the range 300÷500 kg/m³.

According to the Applicant, the soundproofing effects attained by the mixture according to the invention, and by the resulting agglomerate, are extremely favourable, further to the inventiveness of the mixture composition, which contains cellulose inside a cement material combined with “light inerts”.

As far as the laboratory tests are more specifically concerned, they were carried out by using a sample constituted as follows; it is to be noticed that the quantities referred to are the ones needed to obtain 1 m³ of agglomerate:
cement (325) 210 kg
cellulose fibres 10 kg
granular cork 90 lt.
air as much as it is necessary to obtain 1 m³ of agglomerate.
water as much as it is necessary on the basis of the ratio to obtain a fluid agglomerate.

Said sample was characterised by a density of 320 kg/m³ and by a thickness of about 50 mm.

The results obtained were extremely satisfactory, since the tested material noise reduction resulted of 21 dB.

The level reduction of the impact noise of the agglomerate according to the invention was assessed on a test sample; the test was carried out according to the regulations of laboratory internal procedures, by construing the results according to the regulations of rule ISO 717—Part 2.

The sample subjected to the test was formed by the assembly of three slabs of agglomerate, each having the composition and the features mentioned above, of size:

nominal length of the single slab=500 mm;

nominal width of the single slab=500 mm;

nominal thickness of the single slab=about 50 mm.

A sheet of tarred paper, having thickness of 0.5 mm, was located upon the agglomerate slabs.

The sample was placed between a test floor made of reinforced concrete, having thickness of 140 mm, and a screed made of “pietra serena”, having thickness of 40 mm and superficial density of 128 kg/m³.

Said slabs were arranged in a “T” shape and the standardised impact apparatus (Mod. “3204” by Brüel & Kjær) employed for the test was operated in a first horizontal position, along the “T” arm, and subsequently in a second vertical position, along the “T” leg.

The level of generated noise was measured in a receiving chamber, having been characterised through the reverberation time, by means of the spectrum analyser in the frequency range comprised between 100 Hz and 3150 Hz; an analogous measurement was performed by operating the standardised impact apparatus on the screed made of “pietra serena”, directly put in contact with the test floor made of reinforced concrete.

The results evaluation procedure was realised with the aid of the standardised curves according to the rule ISO 717—Part 2, mentioned above.

The comparative analysis of the results quantitatively provided the level reduction of the impact noise due to the contribution of the test sample.

The value of the level of the impact acoustic pressure “L” was calculated, for each frequency band, by using the following formula:
L=Lᵢ+10·log A/A₀

wherein:
Lᵢ=mean level of the acoustic pressure in the receiving chamber, expressed in dB;
A₀=value of the reference surface, equal to 10 m²;
A=equivalent acoustic absorption surface of the receiving chamber, expressed in m², calculated in its turn by using the following formula:
   A=0.16·V/T

wherein:
V=volume of the receiving chamber, expressed in m³₁ equal to 69 m³;
T=reverberation time, expressed in s.

The environmental conditions during the test were the following:

mean environmental temperature=12° C.;

relative humidity=35%;

optimum ventilation.

The test result caused the following evaluation indexes at 500 Hz, within the band of frequencies comprised between 100 Hz and 3150 Hz;

floor+screed: I₁=76 dB

floor+test sample+screed: I₂=55 dB,

thereby the value of noise reduction equal to 21 dB mentioned above.

Afterwards, three additional experimental tests have been carried out at the above-mentioned certified Institute with the same procedure but replacing foam with polystyrene, the samples having the same thickness as above while the corresponding densities were 320 kg/m³ 400 kg/m³ and 480 kg/m³ respectively.

The noise reduction results have proved to be higher than the one of the preceding test, with noise reductions ranging from 24 to 25 dB.

Notwithstanding the invention has been disclosed with reference to preferred constructions, it is in general susceptible of further applications and variations that are intended to be included within the protective scope, as it will be evident for the expert of the field.

In particular, notwithstanding the mixture according to the invention is preferably a cement-based mixture, alternative embodiments of the present invention referring to mixtures based on conventional building binders different from cement, such as lime or clay, are intended to be included within the scope of the present invention.

Furthermore, notwithstanding the method according to the invention provides for the employment of a conventional building concrete-mixer, similar application and results can be achieved in case the mixing occurs in a different equipment and, notably, when the agglomerate instead of being laid onto floor foundations, is employed for producing manufactured articles such as panels, bricks and tiles or for fabricating structural building elements, such as columns, beams and floor slabs, or for other engineering works.

1-30. (canceled)

31. A mixture, containing a building binder and comprising cellulose, ready for preparing water-based hardenable mixings intended to realise mainly soundproofing agglomerates, and wherein, based on the mixture composition, the cellulose amount is comprised between 5 kg and 100 kg for each cubic meter of finished material.

32. A mixture according to claim 31, wherein said finished material has a density comprised between 150 kg/m³ and 2,600 kg/m³.

33. A mixture according to claim 31, wherein said cellulose is in the form of fibres.

34. A mixture according to claim 33, wherein said fibres have a length in the range of 0.05÷10 mm and a diameter in the range of 0.05÷10 mm.

35. A mixture according to claim 31, wherein said cellulose is in the form of paper or cardboard or the like.

36. A mixture according to claim 35, wherein said paper or cardboard or the like is a recycled product.

37. A mixture according to claim 35, wherein the pieces of said paper or cardboard or the like products have size less than 100 mm².

38. A mixture according to claim 31, further comprising cork.

39. A mixture according to claim 38, wherein said cork is in the form of granules or powder.

40. A mixture according to claim 31, further comprising a foam.

41. A mixture according to claim 40, wherein said foam is obtained by transforming a foaming liquid product mixed with water and air.

42. A mixture according to claim 31, further comprising a spongy material.

43. A mixture according to claim 42, wherein said spongy material is polystyrene or similar materials, either virgin or recycled.

44. A mixture according to claim 31, further comprising an inert, called “light inert”, having a specific weight in the range from 0 kg/m³ to about 500 kg/m³.

45. A mixture according to claim 31, further comprising an inert, called “heavy inert”, having a specific weight in the range from about 500 kg/m³ to about 2,000 kg/m³.

46. A mixture according to claim 31, further comprising a coloured pigment.

47. A mixture according to claim 31, wherein the building binder is cement.

48. A mixture according to claim 31, wherein the building binder is lime.

49. A mixture according to claim 31, wherein the building binder is any natural or synthetic binder acting as a cement-like binder.

50. A method for preparing a water-based hardenable mixing intended to realise soundproofing agglomerates comprising the following sequence of steps:
i) supplementing a suitable quantity of water into a building concrete-mixer fit for continues operation;
ii) starting-up the mixer;
iii) inserting a first quantity of cement into the mixer;
iv) inserting a certain quantity of cellulose into the mixer;
v) forcing a certain quantity of air to be introduced inside said mixing by introducing a certain quantity of spongy material or foam into the mixer;
vi) inserting a second quantity of cement into the mixer;
vii) continuously operating said mixer till obtaining a hardenable mixing suitable for laying or for producing manufactured articles.

51. A method according to claim 50, wherein said spongy material is polystyrene.

52. A method according to claim 51, wherein said spongy material is ground polystyrene.

53. A method according to claim 50, further comprising the step of preparing said foam by transforming into foam a foaming liquid product.

54. A method according to claim 50, further comprising the step of preparing said foam by mixing soap with water.

55. A method according to claim 50, wherein said cellulose is in the form of fibres or paper products.

56. A method according to claim 55, wherein said cellulose is in the form of fibres having a length of less than 10 mm and a diameter less than 10 mm.

57. A method according to claim 55, wherein said cellulose is in the form of pieces of paper products having size less than 100 mm².

58. A method according to claim 50, further comprising, between step iv) and step v) in said sequence of steps, the step of inserting a certain quantity of cork, in the form of granules or powder, into the mixer.

59. A method according to claim 58, wherein said cork granules have a size less than 100 mm².

60. A method according to claim 50, further comprising the step of inserting a certain quantity of coloured pigment into the mixer.

61. A method according to claim 50, wherein said articles are panels, bricks or tiles.
